(19) United States
(12) Patent Application Publication
(10) Pub. No.: US 2014/0018682 A1
(43) Pub. Date: Jan. 16, 2014

(54) ULTRASONIC DIAGNOSTIC APPARATUS AND ULTRASONIC DIAGNOSTIC IMAGE RENDERING METHOD

(71) Applicant: Hirotaka Baba, Tokyo (JP)

(72) Inventor: Hirotaka Baba, Tokyo (JP)

(73) Assignee: HITACHI MEDICAL CORPORATION, Tokyo (JP)

(21) Appl. No.: 14/007,841

(22) PCT Filed: Mar. 15, 2012
(86) PCT No.: PCT/JP2012/056618

§ 371 Date: Sep. 26, 2013

(30) Foreign Application Priority Data
Apr. 14, 2011 (JP) 2011-090131

(51) Int. Cl. A61B 8/08; A61B 8/14

(52) U.S. Cl. CPC A61B 8/5207; A61B 8/14
USPC 600/443; 600/437

(57) ABSTRACT

The ultrasonic diagnostic apparatus is equipped with: a gradient calculating section that calculates gradients of the volume data voxel values; a feature calculating section that calculates the feature values of the voxels on the basis of the gradients and the direction of the ultrasonic beam and calculates a feature space on the basis of the feature values; an object-voxel determining section that determines the voxels that correspond to the object on the basis of the feature space; a voxel removing section that removes voxels that are closer to the probe than the object; and an ultrasonic image generating unit that generates ultrasonic images that correspond to the object from the volume data from which the voxels closer to the probe have been removed.

### FIELD OF THE INVENTION

The present invention relates to an ultrasonic diagnostic apparatus, in particular to an ultrasonic diagnostic apparatus and an ultrasonic diagnostic image rendering method for rendering an image of an object to be examined.

### DESCRIPTION OF RELATED ART

When a fetus is rendered using a conventional ultrasonic diagnostic apparatus, the depth of the fetus and a region of interest including the fetus have been manually set for removing the part of which the depth is shallower than the fetus (the part which is closer to the probe than the fetus) from the image. Also for rendering a fetus using a conventional ultrasonic diagnostic apparatus, the setting of the border of a region of interest has been executed by detecting the border of the region of interest using the volume data, detecting and labeling plural voxels in plural borders that are interlined to each other, comparing the labeled voxel groups, and setting the voxels included in the voxel group having the largest number of voxels as the border of the region of interest (for example, see Patent Document 1).

Also in a conventional ultrasonic diagnostic apparatus, the border points between an observation object and a non-observation object have been determined on the basis of the position having the largest luminance gradient in a 2-dimensional image which is selected from the 3-dimensional data (for example, see Patent Document 2).

### PRIOR ART DOCUMENTS

### Patent Documents

Patent Document 1: JP-A-2010-221018

Patent Document 2: JP-A-2006-288471

### SUMMARY OF INVENTION

### Technical Problem

However, since the setting of the border of interest has been executed by detecting the border of the region of interest and setting the border on the basis of the voxel group having the largest number of voxels in the detected border, a huge amount of calculation has been required for rendering an observation target (for example, a fetus) which remains as a problem.

The objective of the present invention is to provide an ultrasonic diagnostic apparatus and an ultrasonic image rendering method capable of rendering a surface image of an object with a small amount of calculation.

### Brief Summary of the Invention

The ultrasonic diagnostic apparatus of the present invention comprises:
a gradient calculating section configured to calculate the gradient of voxel values in the volume data;
a feature calculating section configured to calculate the feature values of the voxels on the basis of the gradients and the direction of the ultrasonic beam, and calculate a feature space on the basis of the feature values;
an object-voxel determining section configured to determine the voxels corresponding to the object on the basis of the feature space;
a volume data processing unit equipped with a voxel removing section configured to remove the voxels that are closer to the probe than the object; and
an ultrasonic image generating unit configured to generate an ultrasonic image corresponding to the object from the volume data from which the voxels positioned on the probe side have been removed.

### Effect of the Invention

In accordance with the present invention, it is possible to provide an ultrasonic diagnostic apparatus and an ultrasonic image rendering method capable of rendering a surface image of an object with a small amount of calculation.

### BRIEF DESCRIPTION OF THE DRAWINGS

FIG. 1 shows the conceptual configuration of an ultrasonic diagnostic apparatus in Embodiment 1.

FIG. 2 shows the configuration of a volume data processing unit 8 in Embodiment 1.

FIG. 3 shows the configuration of an object-voxel determining section 803 in Embodiment 1.

FIG. 4 is a flowchart showing the operation of an ultrasonic diagnostic apparatus in Embodiment 1.

FIG. 5(a) shows the volume data which is represented in the 3-dimensional structure, (b) shows the volume data generated by a volume data generating unit, and (c) shows a cross-section in rθφ-space.

FIG. 6 shows the volume data of a fetus in the uterus.

FIG. 7 is a flowchart showing the operation in which the volume data processing unit identifies a fetal surface.

FIG. 8(a) shows the operand range of operators centering around a target voxel, and (b) shows the operator coefficients to be multiplied by the respective voxel values.

FIG. 9 shows gradient vectors indicated by arrows on a fetal median cross-sectional image.

FIG. 10(a) shows a 3-dimensional feature space representing feature values, and (b) shows the distribution of vector directions of the gradient vectors, (c) shows the distribution of the vector lengths in the gradient vectors, (d) shows the distribution of vector directions of the gradient vectors after filtering, and (e) shows the distribution of victor lengths in the gradient vectors after filtering.

FIG. 11(a) shows the distribution in the distribution region selected by a filtering part, (b) shows the frequency distribution in the distribution regions categorized by the depth of the voxels, and (c) is a view showing that the variance value is calculated for each cluster by a cluster selecting part.

FIG. 12 is a fetal median cross-sectional image in the condition in which the voxels that are closer to a probe than a fetal surface are removed.

FIG. 13 shows the configuration of an object-voxel determining section in Embodiment 2.

FIG. 14(a) shows the distribution of vector lengths and vector directions in a feature space, (b) shows the frequency distribution of the vector lengths categorized by the vector direction, and (c) shows the frequency distribution of vector directions categorized by the vector length.

FIG. 15 shows a volume data processing unit in Embodiment 3.

FIG. 16 shows the operand range of operators adjusted by the operation unit.

FIG. 17 is a view showing that the operand range of operators is variable.

### DETAILED DESCRIPTION OF THE INVENTION

The ultrasonic diagnostic apparatus related to the present embodiment comprises:
a volume data generating unit configured to generate the volume data of an object by transmission/reception of ultrasonic beams from/by a probe; and
a volume data processing unit configured to generate an ultrasonic image of the object which is generated by the volume data generating unit; and
an ultrasonic image generating unit configured to generate the ultrasonic image corresponding to the object,
wherein the volume data processing unit is equipped with:
a gradient calculating section configured to calculate the gradients of the voxel values in the volume data;
a feature calculating section configured to calculate the feature values of the voxels on the basis of the gradient and the direction of the ultrasonic beam and to calculate a feature space on the basis of the feature values;
an object-voxel determining section configured to determine the voxels corresponding to the object on the basis of the feature space; and
a voxel removing section configured to remove the voxels that are closer to the probe than the object.

In accordance with such configuration, by generating an ultrasonic image from the voxels determined based on the direction of the ultrasonic beam and the gradients of the voxel values, the gradients of the voxel values are characterized based on the direction of the ultrasonic beam and the feature values which represent the feature of the voxels are calculated for determining the voxels of the object based on the feature space of the feature values, thereby making it possible to render a surface image of the object.

Also, while a conventional ultrasonic diagnostic apparatus detects the border of a region of interest and sets the border of the region of interest on the basis of the voxel group having the largest number of voxels in the border, when the borders such as between the fat and the uterus or between the fetal myelocoel and the region of which the depth is deeper than the fetus that are interlinked to each other becomes large, it can solve the problem of difficulty in distinguishing the border of the region of interest.

Also, while the conventional ultrasonic diagnostic apparatus sets the border points on the basis of the position having the largest luminance gradient in the 2-dimensional cross-sectional image, the present embodiment can solve the problem of misidentifying a non-fetal surface region as a fetal surface region, when the border is observed on the basis of only the position having the largest luminance gradient in the cross-sectional image extracted from the 3-dimensional image and the luminance gradient is larger than that of the fetal surface, for example in the case that multiple echo is generated or the border between the fat and the uterus exists.

Also, while there is a method of clustering the voxels equivalent to an object by the averaging method, etc. which uses the barycenter of the voxel values for rendering the image of the object, the present embodiment can solve the problem of difficulty in rendering an image of an object in real time due to a huge amount of calculation required by the clustering method.

Also in the present embodiment, the object-voxel determining section comprises a cluster selecting part configured to determine the voxels including the object on the basis of the distribution of the vector length and/or the vector direction of the gradient in the feature space.

In accordance with such configuration, a surface image of an object can be rendered with a small amount of calculation, since the voxels corresponding to the object is determined from the distribution of the vector length or the vector direction of the gradient in the feature space.

Also, the present embodiment is characterized in that the vector direction of the cluster selecting part is expressed by the inner product of the normalized victor of the ultrasonic beam and the normalized vector of the gradient of the voxel values in the volume data.

In accordance with such configuration, the feature values which represent the feature of the voxels are calculated by the inner product of the normalized vector of the ultrasonic beam and the normalized vector of the gradient, whereby making it possible to render a surface image of an object with a small amount of calculation.

The present embodiment is also characterized in that the distribution in the cluster selecting part is the frequency distribution of the vector lengths or the vector directions categorized by the depth, wherein the index of the distribution is represented by at least one of the variance value, standard deviation and average deviation on the basis of the frequency distribution.

By such configuration, the voxels including the object are determined by the variance values, standard deviation or average deviation on the basis of the frequency distribution of the vector lengths or the vector directions, whereby the surface image of an object can be rendered with a small amount of calculation.

The present embodiment is also characterized in that the object-voxel determining section determines the voxels including the object by comparing the preset threshold value and the feature values.

In accordance with such configuration, the feature values can be easily distinguished by the threshold value, whereby the surface image of an object can be rendered with a small amount of calculation.

Also, the present embodiment is characterized in that the object-voxel determining section comprises a distribution calculating unit configured to calculate the distribution of the vector lengths and/or the vector directions in the feature space, and a threshold value determining unit configured to determine the threshold value on the basis of the calculated distribution.

In accordance with such configuration, the threshold value to be used in the filtering part can be determined on the basis of the distribution of the vector lengths or the vector directions in a feature space.

The present embodiment is also characterized in that the feature calculating section calculates the feature space having the feature values of at least one of the vector lengths of the gradient, the vector direction of the gradient and the depth of the voxels in the volume data voxel values.

In accordance with such configuration, since the feature values which represent the feature of the voxels is calculated from at least one of the vector lengths of the gradient, the vector directions of the gradient and the depth so as to distinguish an object on the basis of the feature space in which the previously mentioned feature values are set as each axis, the surface image of the object can be rendered.

Also, the present embodiment is characterized in that the voxel removing section sets the voxel value of the voxels that are positioned on the probe side as a predetermined value.

In accordance with such configuration, the voxels that are closer to the probe than an object can be removed by setting a predetermined value on the voxels that are positioned on the probe side, thus the surface image of the object can be rendered.

The present embodiment also is characterized in that the voxel removing section sets the transparency on the voxels that are positioned on the probe side.

In accordance with such configuration, by setting the transparency of the voxels that are closer to the probe than an object, the voxels that are on the probe side can be removed and the surface image of the object can be rendered.

The present embodiment is also characterized in that the gradient calculating section calculates the gradient in three dimensions on the basis of operators, and the operand range of the operators is variable.

Also, the present embodiment comprises a device for setting the operand range of the gradient in three dimensions, wherein the gradient calculating section calculates the gradient in three dimensions on the basis of the set operand range.

In accordance with any of the above-described configuration, it is possible to remove the noise on an object surface and to render a smooth surface image of the object with a small amount of calculation by variably setting the operand range.

The ultrasonic image rendering method related to the present embodiment generates an ultrasonic image of an object from the volume data obtained by the ultrasonic diagnostic apparatus having a probe, and includes:
a step of calculating the gradient of the voxel values of the volume data;
a step of calculating the feature values of the voxels on the basis of the vector directions of the gradient and the gradients of the voxel values so as to calculate the feature space on the basis of the feature values;
a step of determining the voxels corresponding to the object on the basis of the feature space;
a step of removing the voxels that are closer to the probe than the object; and
a step of generating an ultrasonic image corresponding to the object from the volume data from which the voxels that are positioned on the probe side have been removed.

The present embodiment is also characterized in that the step for determining the voxels comprises a cluster selecting step which determines the voxels including the object based on the distribution of the vector lengths and/or the vector directions of the gradients in the feature space.

The present embodiment is also characterized in that the step of determining the voxels determines the voxels including the object by comparing the preset threshold value and the feature values.

Also, the present embodiment is characterized in that the step of calculating the feature space calculates a feature space in which at least one of the vector-length and the vector-direction of the gradient in the voxel values in the volume data and the depth of the voxels is set as the feature value.

In accordance with any of the above-described configurations, by generating an ultrasonic image from the voxels determined on the basis of the direction of the ultrasonic beam and the gradient of the voxel values, the gradient of the voxel values are characterized by the direction of the ultrasonic beam and the feature values which represent the feature of the voxels are calculated so as to determine the voxels of an object on the basis of the feature space of the feature values, thereby the surface image of the object can be rendered.

### Embodiment 1

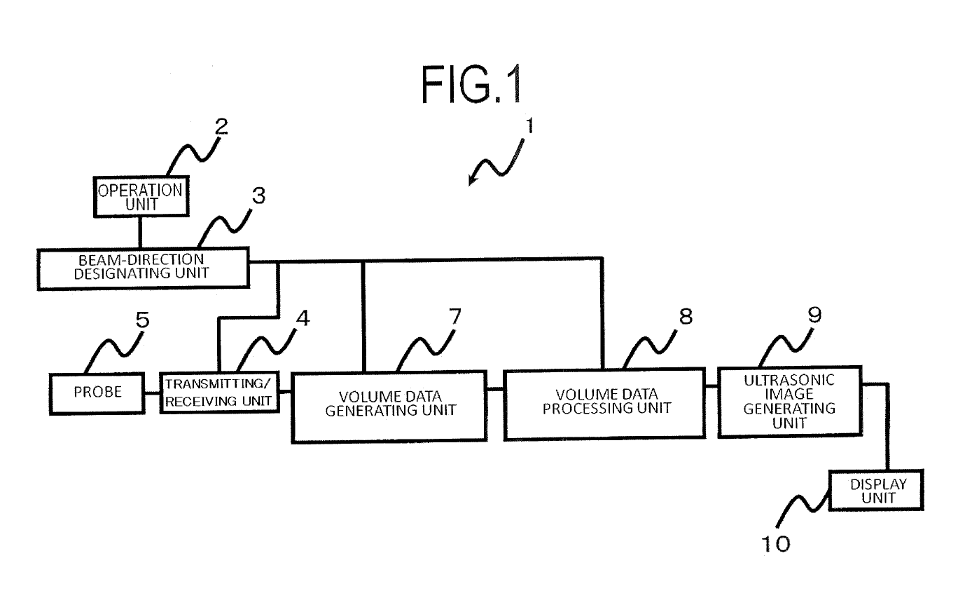

The ultrasonic diagnostic apparatus in Embodiment 1 of the present embodiment will be described below referring to the attached diagrams. FIG. 1 shows the conceptual configuration of an ultrasonic diagnostic apparatus in the present embodiment.

An ultrasonic diagnostic apparatus 1 comprises an operation unit 2, a beam-direction instructing unit 3, a transmitting/receiving unit 4, a probe 5, a volume data generating unit 7, a volume data processing unit 8, an ultrasonic image generating unit 9 and a display unit 10.

The operation unit 2 performs the operation of the ultrasonic diagnostic apparatus 1, executes various setting for rendering a 3-dimensional image of an object, and instructs the rendering of the 3-dimensional image of the object. The operation unit 2 also instructs the direction of the ultrasonic beam to an ultrasonic-beam direction instructing unit. The direction of the ultrasonic beam is transmitted to the volume data generating unit 7 and the volume data processing unit 8 as the data.

The transmitting/receiving unit 4 generates transmission signals of the ultrasonic beam irradiated in the direction of the ultrasonic beam which is instructed by the operation unit 2. The transmitting/receiving unit 4 transmits the generated transmission signal to the probe 5, and receives the reception signal from the probe 5. Also, transmitting/receiving unit 4 comprises a transmission circuit, transmission delay circuit, reception circuit, reception delay circuit, etc. as disclosed in JP-A-2001-252276.

The probe 5 converts the transmission signal transmitted from the transmitting/receiving unit 4 into an acoustic signal, and irradiates the ultrasonic beam to the object via a medium. Also, the probe 5 converts the reflected echo signal reflected in the object into a reception signal, and transmits the converted signal to the transmitting/receiving unit 4.

The volume data generating unit 7 receives the reception signal received by the probe 5 from the transmitting/receiving unit 4, and generates the volume data of the object on the basis of the reception signals.

Also, the volume data generating unit 7 associates the direction of the ultrasonic beam with the voxel values, and generates the volume data.

The volume data processing unit 8 processes the volume data generated by the volume data generating unit 7, and transmits the 3-dimensional image data of the target area in the object to the ultrasonic image generating unit 9 as an image projected on a 2-dimensional plane.

The ultrasonic image generating unit 9 generates an ultrasonic image on the basis of the image data received from the volume data processing unit 8. The display unit 10 displays an ultrasonic image generated by the ultrasonic image generating unit 9.

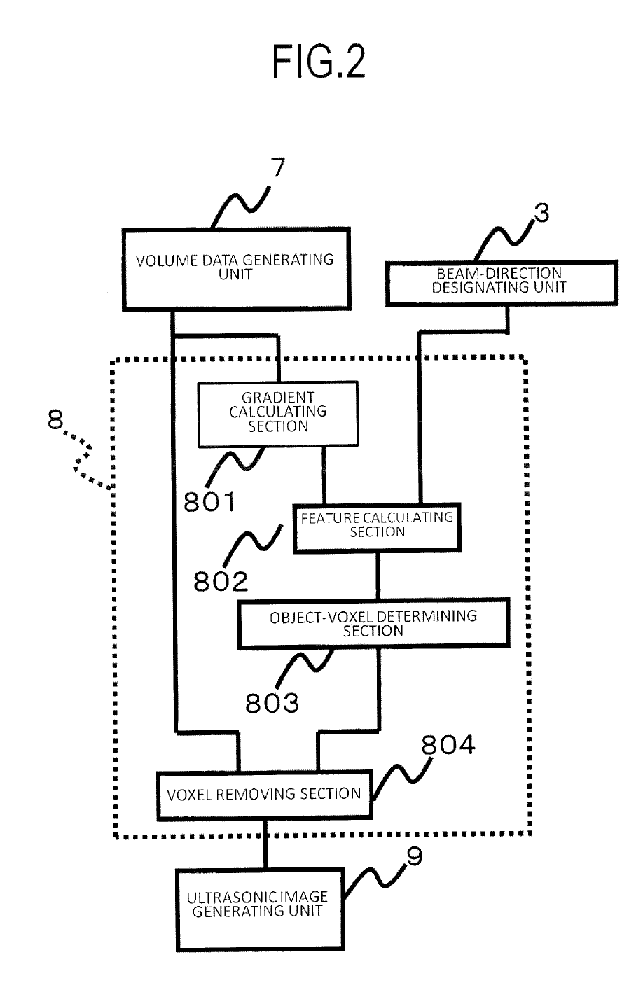

FIG. 2 shows the configuration of the volume data processing unit 8 in the present embodiment. As shown in FIG. 2, the volume data processing unit 8 comprises a gradient calculating section 801, a feature calculating section 802, an object-voxel determining section 803 and a voxel removing section 804.

The gradient calculating section 801 calculates the gradient of the voxel values in the volume data generated by the volume data generating unit 7. The gradient calculating section 801 respectively calculates the gradient of the voxel values in each axis-direction of the 3-dimensional coordinates, and calculates the gradient vectors in three dimensions (3-dimensional gradients).

The feature calculating section 802 receives the direction of the ultrasonic beam from the beam-direction instructing unit 3. The feature calculating section 802 receives the 3-dimensional gradients from the gradient calculating section 801, and calculates the lengths and the directions of the gradient vectors on the basis of the gradients in each axis-direction of the 3-dimensional coordinates.

Also, the feature calculating section 802 calculates the normalized gradient vector of which the gradient vector length is 1 (the normalized vector of the gradient) for each voxel. The feature calculating section 802 calculates the normalized beam vector of which the beam vector length of the ultrasonic beam is 1 (the normalized vector of an ultrasonic beam) for each voxel. The feature calculating section 802 calculates the inner product of the normalized victor of the ultrasonic beam and the normalized vector of the gradient.

In other words, the feature calculating section 802 calculates the feature values of the voxels having the voxel value on the basis of the ultrasonic-beam direction and the gradient of the voxel values, and calculates the feature space along with the depth of the voxels.

The object-voxel determining section 803 receives from the feature calculating section 802 the feature space having the feature value of at least one of the gradient vector lengths, the gradient vector directions and the depth of the voxels. The object-voxel determining section 803 distinguishes an object (for example, a fetal surface) on the basis of a feature space, and determines the voxels corresponding to the object. The object-voxel determining section 803 transmits the coordinates of the determined voxels to the voxel removing section 804.

The voxel removing section 804 removes the voxels of the coordinate values that are shallower than the voxel coordinate value of an object (the voxels that are closer to the probe than the object) from the volume data, and transmits the volume data from which the voxels have been removed to the ultrasonic image generating unit 9.

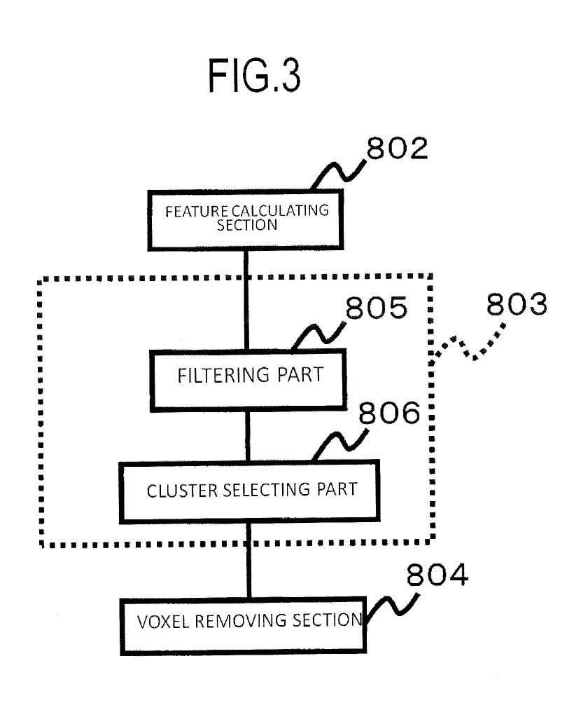

FIG. 3 shows the configuration of the object-voxel determining section 803 in the present embodiment. As shown in FIG. 3, the object-voxel determining section 803 comprises a filtering part 805 and a cluster selecting part 806. The object-voxel determining section 803 determines the voxels corresponding to an object by comparing a preset threshold value and the feature value using the filtering part 805. For example, the filtering part 805 selects the feature value larger than the threshold value as the feature value of the object, and transmits the selected value to the cluster selecting part 806.

The cluster selecting part 806 calculates the distribution of the gradient vector lengths or the gradient vector directions with respect to the depth of the voxels on the basis of the feature space. The index of the distribution (variability, etc.) is represented by the variance values. For example, the cluster selecting part 806 counts the frequency of the gradient vector lengths or the gradient vector directions categorized by the depth of the voxels, divides the measured frequencies into plural clusters on the basis of the frequency distribution, and calculates the variance value for each cluster.

The cluster selecting part 806 determines the voxels corresponding to an object by comparing a preset threshold value and the index of the distribution. For example, the cluster selecting part 806 selects the cluster having the variance values that are greater than a predetermined value. The cluster selecting part 806 determines the voxels corresponding to an object on the basis of the depth of the voxels. For example, the cluster selecting part 806 determines, from among the clusters having the variance values that are greater than a predetermined threshold value, the voxels having the shallowest average value in the depth of the cluster as the voxels corresponding to the object, and transmits the coordinates of the determined voxels to the voxels removing unit 804.

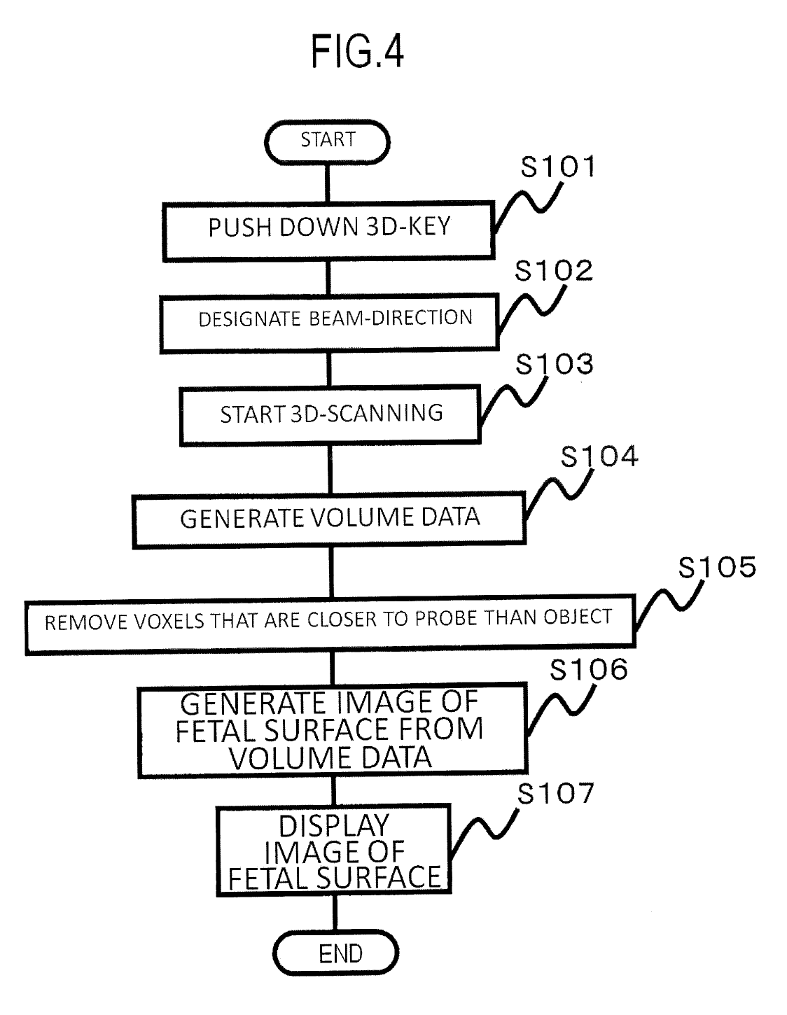

Next, the operation of an ultrasonic diagnostic apparatus in the present embodiment will be described. FIG. 4 is a flowchart showing the operation of an ultrasonic diagnostic apparatus in the present embodiment. A case in which a fetal surface in the uterus is displayed as an object will be described in the present embodiment.

A user of the ultrasonic diagnostic apparatus applies the probe 5 on an object, and renders a median cross-sectional image (sagittal image) of a fetus in the uterus by 2-dimensional ultrasonic scanning. Then the user determines the direction of the probe 5 for 3-dimensional scanning on the basis of the median cross-sectional image, and a 3-dimensional key in the operation unit 2 is pushed down (step S101).

In this case, the information that the 3-dimensional key is pushed down is transmitted to the beam-direction instructing unit 3, and the beam-direction instructing unit 3 transmits the direction of the ultrasonic beam for 3-dimensional scanning to the transmitting/receiving unit 4, volume data generating unit 7, volume data processing unit 8 and ultrasonic image generating unit 9 (step S102).

The transmitting/receiving unit 4 receives the direction of the ultrasonic beam, and generates the transmission signal of the ultrasonic beam to be irradiated in the instructed direction of the ultrasonic beam. The probe 5 starts the 3-dimensional scanning of the object on the basis of the generated transmission signal (step S103).

The probe 5 transmits the reception signal to the volume data generating unit 7 via the transmitting/receiving unit 4, and the volume data generating unit 7 arranges the reception signal (reception echo) of the ultrasonic beam as the voxel value in the instructed ultrasonic beam direction and generates the volume data of the object (step S104).

The volume data processing unit 8 distinguishes the fetal surface on the basis of the generated volume data, removes the voxels that are closer to the probe than the fetal surface from the volume data, and transmits the volume data from which the voxels have been removed to the ultrasonic image generating unit 9 (step S105).

The ultrasonic image generating unit 9 generates an image of the fetal surface which is projected on the 2-dimensional plane on the basis of the volume data from which the voxels that are closer to the probe than the fetal surface have been removed, and transmits the image of the fetal surface to the display unit 10 (step S106). The display unit 10 displays the image of the fetal surface (step S107).

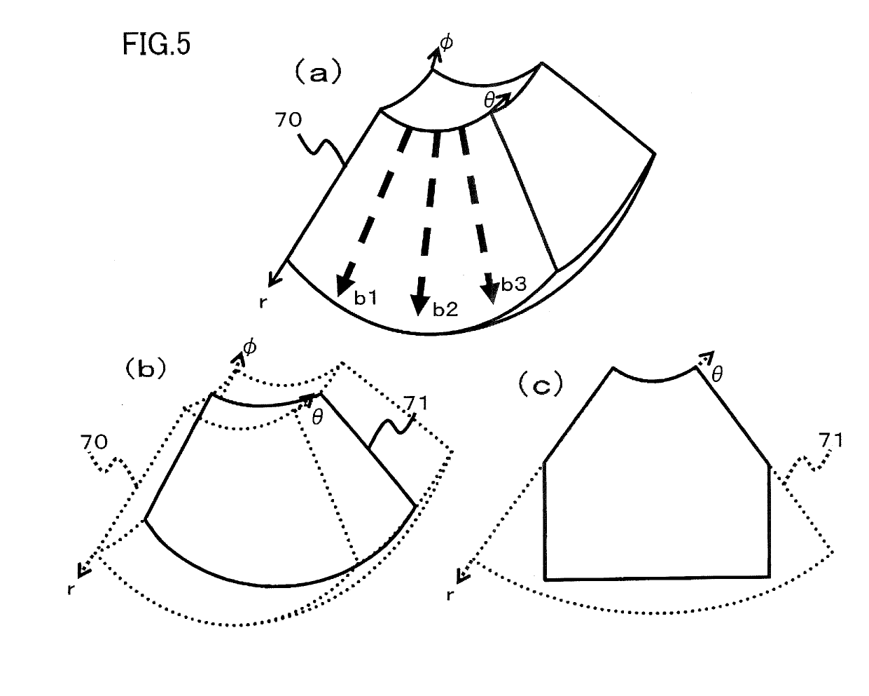

Next, the volume data which is generated by the volume data generating unit 7 in step S104 of FIG. 4 will be described referring to FIG. 5. As shown in FIG. 5(a), the volume data generating unit 7 generates the volume data which is represented in three dimensions. Ultrasonic beams b1, b2 and b3 are respectively irradiated in scanning performed using the probe 5, and the volume data generating unit 7 generates the volume data by setting the depth direction of the ultrasonic beam as r-axis and the scan direction of the ultrasonic beam as θ-axis and φ-axis. The volume data generating unit 7 arranges the reception signal of the ultrasonic beam as data in r-axis direction (ultrasonic-beam direction) in accordance with θ-axis and φ-axis in the scan direction, and forms rθφ-space 70 as shown in FIG. 5(a). Also, the volume data of an arbitrary cross-section 71 is extracted from the rθφ-space 70 on the basis of the volume data generated by the volume data generating unit 7 as shown in FIG. 5(b), and a partial region (solid-line part) in the cross-section 71 in the rθφ-space 70 is displayed on the display unit 10 as shown in FIG. 5(c).

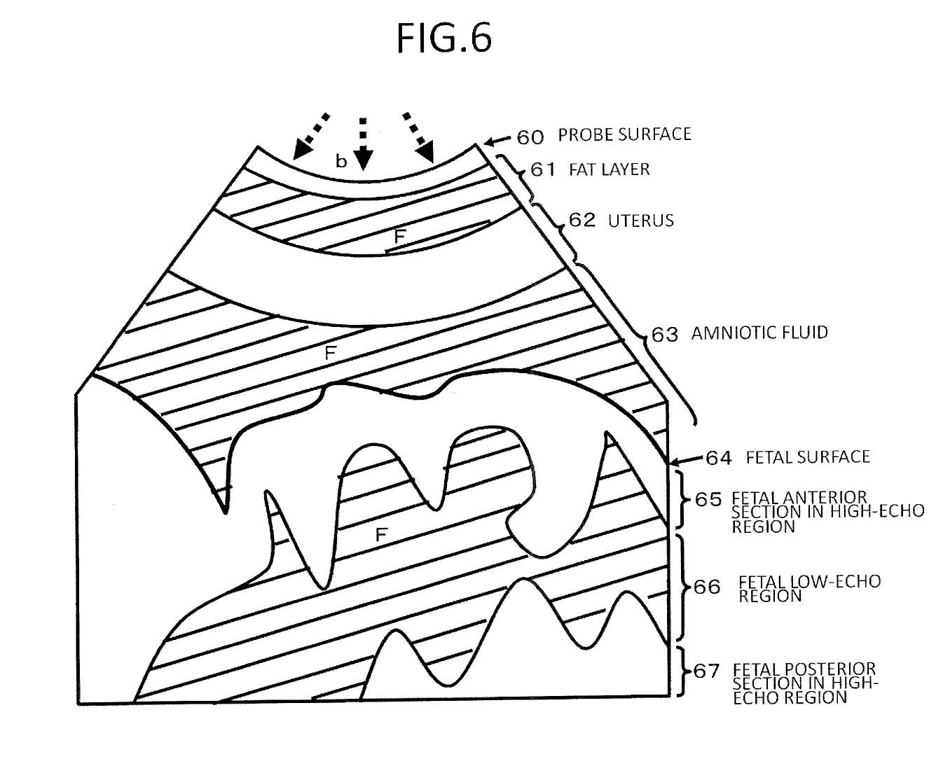

Next, the operation in step S105 will be described in which the volume data processing unit 8 distinguishes the surface of a fetus and removes the voxels that are closer to the probe than the fetal surface from the volume data. FIG. 6 shows the volume data of a fetus in the uterus. While a 3-dimensional image projected on a 2-dimensional plane is generally represented on the basis of the volume data in three dimensions, a median cross-sectional image of a fetus in the uterus will be shown here for the illustrative purpose.

As shown in FIG. 6, along the depth direction of the ultrasonic beams b, a probe surface 60, a fat layer 61, a uterus 62, amniotic fluid 63, a fetal surface 64, a fetal anterior section in high-echo region 65, a fetal low echo region 66, and a fetal posterior section in high echo region 67 are generated by the volume data generating unit 7 as the volume data. Regions F denoted by oblique lines in FIG. 6 have weak reflected echo signals with low luminance which are displayed darkly (low-echo regions), and the regions without oblique lines have strong reflected echo signals with high luminance which are displayed brightly (high-echo regions). The uterus 62, the fetal anterior section in high-echo region 65 and the fetal posterior section in high-echo region 67 are high-echo regions, and the fat layer 61, the amniotic fluid 63 and the fetal low-echo region 66 are low-echo regions. The volume data processing unit 8 distinguishes the fetal surface 64 which is the border between the amniotic fluid 63 and the fetal anterior section in high-echo region 65, and determines the voxels corresponding to the fetal surface 64 from the volume data.

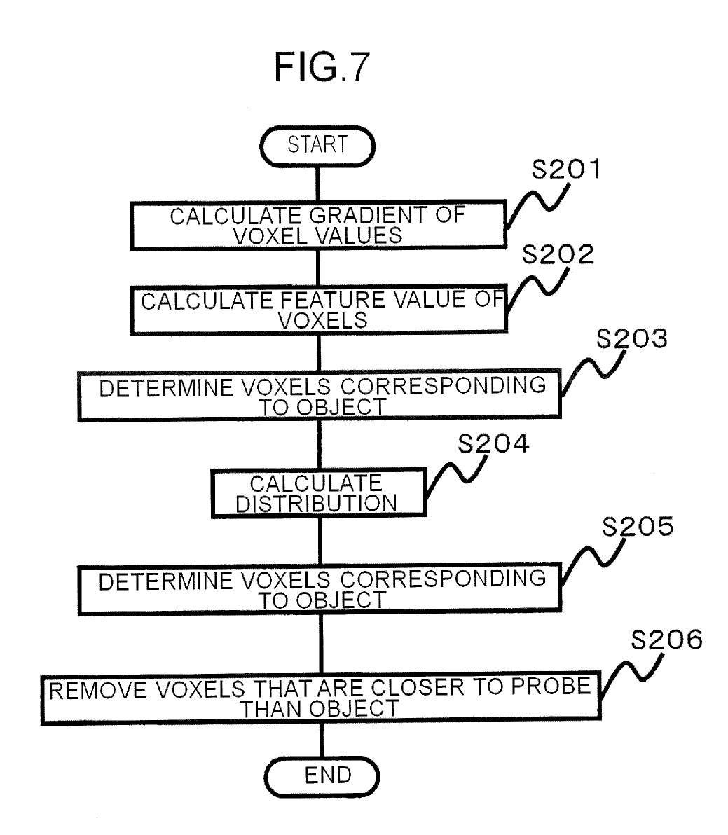

FIG. 7 is a flowchart showing the operation in which the volume data processing unit 8 distinguishes the fetal surface 64. The gradient calculating section 801 calculates the gradient of the voxel values in the volume data using operators (step S201). A known operator such as the Prewitt or Sobel may be used for calculating the gradient. Here, simple operators are used for illustrative purpose.

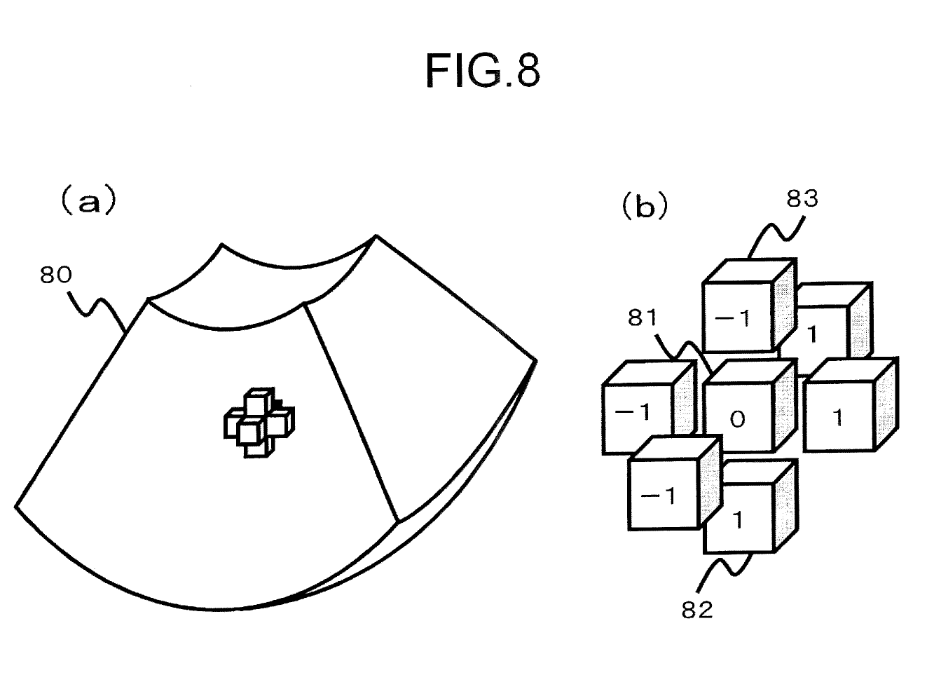

FIG. 8(a) is a view showing the operand range of operators centering around predetermined target voxels in volume 80. FIG. 8(b) is a view showing the operator coefficients to be multiplied by the respective voxels. As shown in FIG. 8(b), the gradient calculating section 801 calculates the gradient of the target voxels by setting three voxels in each coordinate-axis direction (the front and back, right and left, and above and below) as the operand range. The gradient calculating section 801 multiplies the voxel values of each calculation target by the operator coefficient and sums up the multiplication results for each coordinate axis, and calculates the totalized value as the gradient of each coordinate axis. For example in FIG. 8(b), when the gradient of the vertical coordinate-axis is calculated, the voxel value of a target voxel 81 is multiplied by operator coefficient “0”, the voxel value of a voxel 82 is multiplied by the operator coefficient “1”, the voxel value of a voxel 83 is multiplied by operator coefficient “−1”, and the totalized value of the previously multiplied values is recorded as the gradient of the vertical coordinate-axis. In the same manner, the gradients of the other two coordinate-axes are calculated. Then by shifting the target voxel to the adjacent voxel and repeating the same calculation, the gradient of the entire volume is calculated for each coordinate axis. Accordingly, the gradients of the respective voxels in the volume data are calculated by the gradient calculating section 801, and the gradients become the vectors having the components in each coordinate-axis direction (3-dimensional gradients).

In a case in which the gradient of a target voxel is calculated by operators shown in FIG. 8(b), if all the voxels adjacent to a target voxel have the same voxel value, all of the respective coordinate-axis direction components of the gradient become “0”. On the other hand, if the voxels adjacent to a target voxels have different voxel values along a predetermined coordinate direction (for example, the vertical coordinate-axis direction) and all of the adjacent voxels in the other coordinate-axis directions have the same voxel value, the gradients become the vectors which have the components only in the predetermined coordinate-axis direction (vertical coordinate-axis direction). In this manner, the gradient calculating section 801 calculates the 3-dimensional gradients as the gradient vectors.

Next, the feature calculating section 802 calculates the inner product of the normalized vector of the gradient vector length, gradient vector direction and the ultrasonic beam and the normalized vector of the gradient as the feature value of the voxels, on the basis of the 3-dimensional gradients received from the gradient calculating section 801 (step S202).

The object-voxel determining section 803 distinguishes the fetal surface on the basis of the feature value which is calculated by the feature calculating section 802, and determines the voxels corresponding to the fetal surface (step S203).

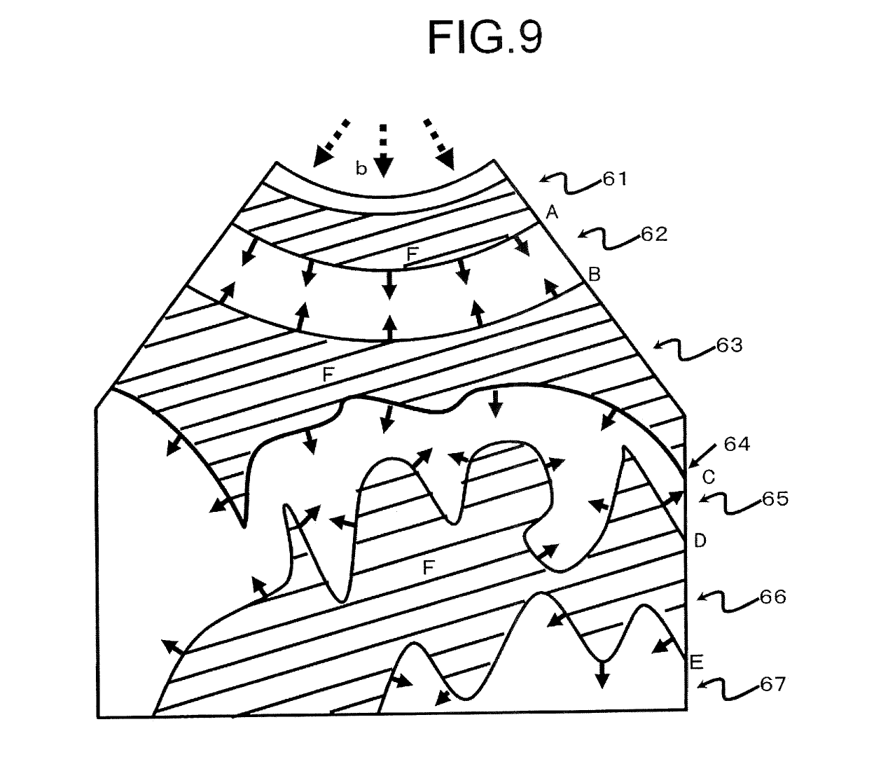

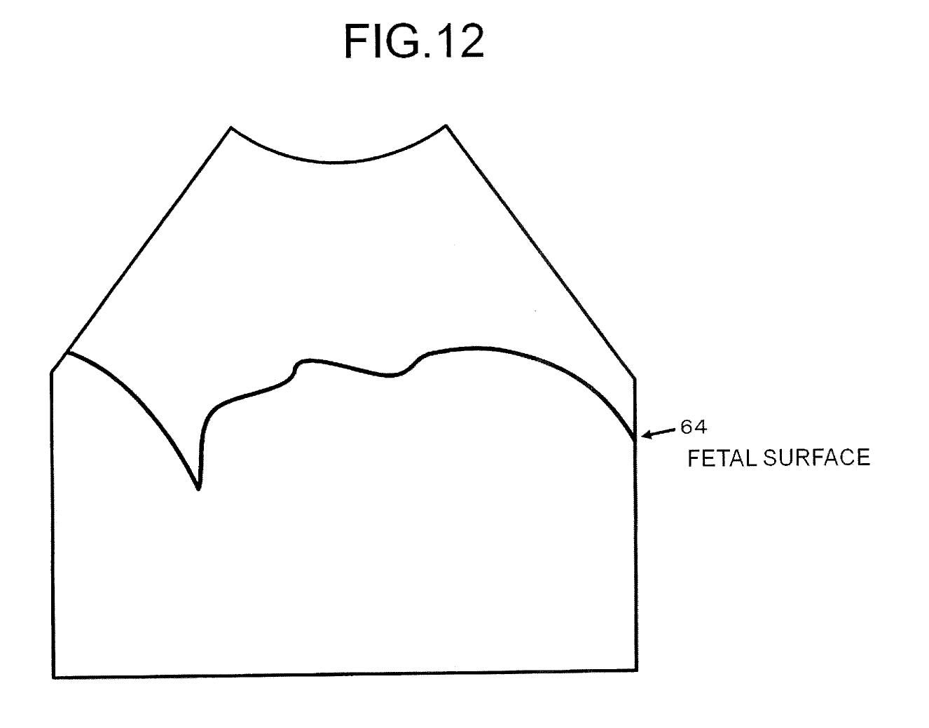

The operation of the object-voxel determining section 803 will be described referring to FIG. 9˜FIG. 12. FIG. 9 is a median cross-sectional image of a fetus on which the gradient vectors are denoted by arrows. While the gradient is usually calculated for all voxels in the volume, only the gradient vectors with large length are mainly indicated in the diagram for illustrative purpose. The lengths of the arrows indicate the gradient vector lengths, and the directions of the arrows indicate the gradient vector directions.

As shown in FIG. 9, the portions with long gradient vectors are a border A between the fat layer 61 and the uterus 62, a border B between the uterus 62 and the amniotic fluid 63, a border C between the amniotic fluid 63 and the fetal anterior section in high-echo region 65, a border D between the fetal anterior section in high-echo region 65 and the fetal low-echo region 66 and a border E between the fetal low-echo region 66 and the fetal posterior section in high-echo region 67. The directions of the vectors in border A and border B are about the same as ultrasonic beam b (i.e., variability is comparatively small), but the vector directions are opposite. The vector directions in Border A are the depth direction, and the vector directions in border B are the opposite to the depth direction. The vector directions of the gradient vectors in border C and border E are about the same as the direction of ultrasonic beam b (depth direction), but the directions are varied (i.e., variability is comparatively great). The vector direction of the gradient vectors in border D is on the probe side (opposite to the depth direction), but the directions are varied (i.e., variability is comparatively great). The gradient vectors of region F besides borders A˜F (not shown in the diagram) have shorter vector lengths compared to the gradient vectors in borders A˜E, and the variability in vector directions is great.

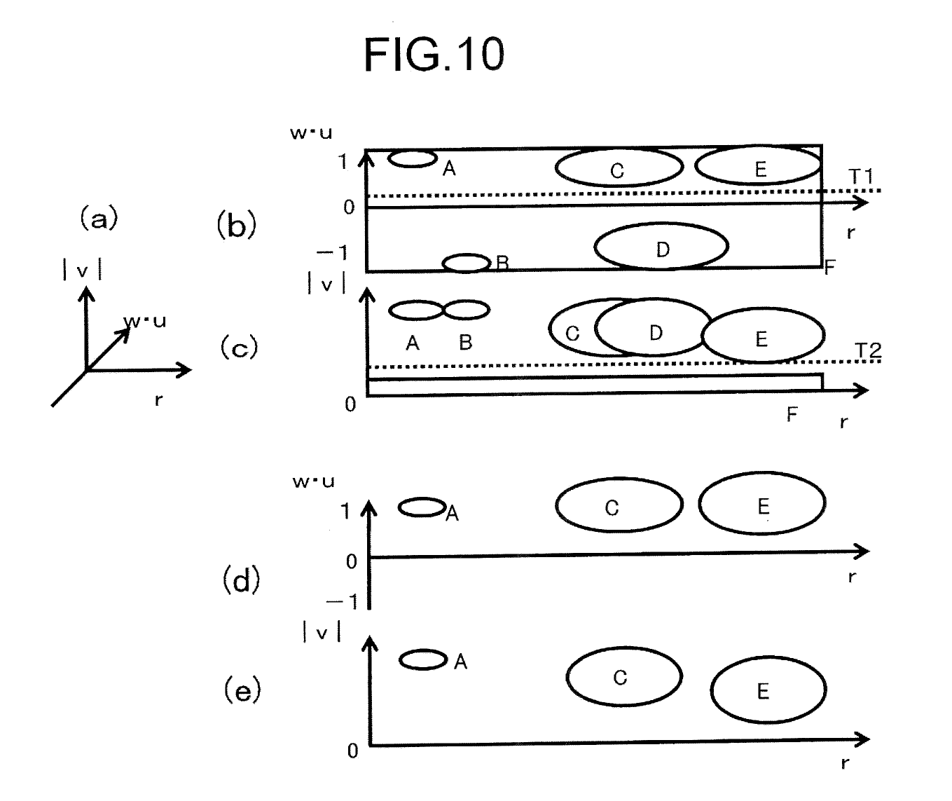

FIG. 10 shows the distribution of the vector lengths and vector directions of the gradient vectors with respect to the voxel depth. FIG. 10(a) shows a 3-dimensional feature space representing the feature values (the vector length is denoted by |v|, the vector direction is denoted by w·u, and the voxel depth is denoted by r). The vector direction is represented by the vector direction with respect to the direction of ultrasonic beam b, and concretely expressed by the inner product w·u of the normalized vector of ultrasonic beam b and the normalized vector of the gradient. In w·u, w is the unit vector of ultrasonic beam b (normalized beam vector), and u is the normalized gradient vector which is normalized by dividing gradient vector v by the gradient vector length |v|.

FIG. 10(b) shows the distribution of vector direction w·u of the gradient vector with respect to the voxel depth r. FIG. 10(c) shows the distribution of vector length |v| of the gradient vector with respect to the vector depth r. While the feature values are represented by the 3-dimensional feature space of vector length |v|, vector direction w˜u and depth r, they are divided for illustrative purpose in the diagram into vector direction w·u and vector length |v| with respect to voxel depth r. As shown in FIG. 10(b), vector directions w·u with respect to voxel depth r are distributed, and vector directions w·u in borders A˜E and region F shown in FIG. 9 are distributed respectively in distribution regions A˜F. Since region F shown in FIG. 9 has large variability of vector directions w·u compared to borders A˜E, distribution region F is distributed overall as shown in FIG. 10(b). On the other hand, as shown in FIG. 10(c), vector lengths |v| with respect to depth r are distributed, and vector lengths |v| in borders A˜E and region F shown in FIG. 9 are distributed respectively in distribution regions A˜F. Since region F shown in FIG. 9 has weak reflected echo signals with low luminance compared to borders A˜E, distribution region F is distributed with small values as shown in FIG. 10(c).

The object-voxel determining section 803 distinguishes the fetal surface 64 (border C) on the basis of the feature values, and determines the voxels of the distribution region in border C. There is a conventional technique referred to as clustering for specifying the distribution region of a border. However, when volume data in a 3-dimensional feature space is performed with the clustering using the conventional technique, it takes a long period of time for the clustering process. Therefore in the present embodiment, the method in which the object-voxel determining section 803 determines the voxels corresponding an object by comparing a present threshold value and the feature value and the method of determining the voxels corresponding to an object by comparing a present threshold value and the index of distribution (variability) are used for rendering border C (the surface image of an object) with a small amount of calculation.

The object-voxel determining section 803 determines the voxels corresponding to an object by comparing a preset threshold value and the feature value using the filtering part 805 (step S203). As shown in FIG. 10(b), when a preset threshold value of vector direction w·u is set as T1, the filtering part 805 performs filtering and selects the distribution in the region having the value of vector direction w·u which is larger than preset threshold value T1. As a result of filtering on the basis of threshold value T1, a part of distribution region F and distribution regions A, C and E are selected. Also as shown in FIG. 10(c), when a preset threshold value of vector length |v| is set as T2, the filtering part 805 performs filtering and selects the distribution in the region of vector length |v| having the value larger than threshold value T2. As a result of filtering on the basis of threshold T2, distribution regions A, C and E are selected. In other words, when the filtering part 805 performs filtering on the basis of threshold value T1 and threshold value T2, distribution regions A, C and E are selected and unnecessary borders B, D and region F are removed using the feature values in the borders as shown in FIGS. 10(d) and (e).

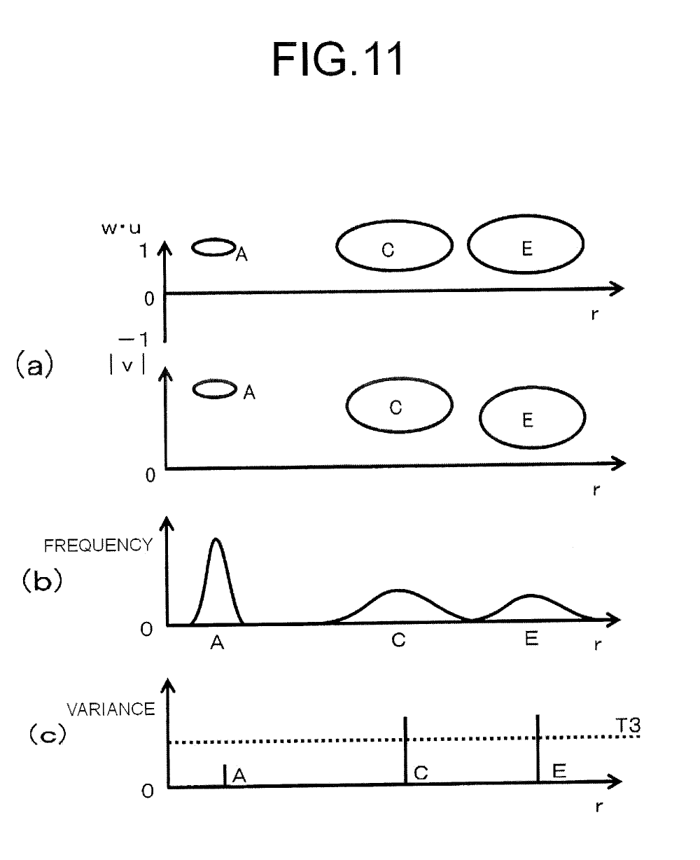

The cluster selecting part 806 included in the object-voxel determining section 803 calculates the index (variability) of the distribution in vector length |v| or vector direction w·u with respect to the voxel depth (step S204). FIG. 11(a) shows the distribution of distribution regions A, C and E selected by the filtering part 805. FIG. 11(b) is the frequency distribution of distribution regions A, C and E categorized by the depth of voxels. Either one of vector length |v| and vector direction w·u with respect to the voxel depth may be used for the frequency distribution.

As shown in FIG. 11(b), the cluster selecting part 806 distinguishes each frequency distribution of distribution regions A, C and E. In order to distinguish the frequency distribution of the distribution regions respectively, the inclination of the curve in the frequency distribution may be calculated by the first-order differential, etc. and the places in which the inclination changes from the negative to the positive can be used as a border.

As for the inclination of the frequency distribution, the inclination of a straight line by which the frequencies for each class are connected may be used, or the inclination of the curve in which the smoothing process is executed on the frequency distributions connected by a straight line may be used.

As shown in FIG. 11(c), the cluster selecting part 806 divides the frequency distribution into plural clusters (clusters of distribution regions A, C and E) by distinguishing the frequency distribution in each of distribution regions A, C and E, and calculates the variation values based on the frequency distribution of each cluster. Since depth r in the ultrasonic beam direction of order A between the fat layer 61 and the uterus 62 shown in FIG. 9 is approximately constant compared to borders C and E, the variance value in distribution region A corresponding to border A is small compared to the other distribution regions C and E. Therefore, when a preset threshold value is set as T3, the cluster selecting part 806 selects the clusters having the variance value which is larger than threshold value T3 (distribution regions C and E) as shown in FIG. 11(c). Further, the cluster selecting part 806 determines the cluster which has the shallowest average value of depth r in the cluster from among the selected clusters (distribution region C) as the voxels corresponding to the fetal surface 65 (border C) (step S205). That is, the cluster selecting part 806 selects distribution region C on the basis of threshold value T3 and depth r, then removes unnecessary borders A and E.

FIG. 12 is a fetal median cross-sectional image from which border C is selected and the voxels in border C (the voxels closer to the probe than the fetal surface) have been removed. As shown in FIG. 12, the voxel removing section 804 removes the voxels having the coordinate values which are shallower than that of the voxels in border C corresponding to the selected distribution region C from the volume data (step S206). In addition, any method for removing the voxels from the volume data may be used which is appropriate for the operation of the ultrasonic image generating unit 9. For example, when the maximum value projection method is used by the ultrasonic image generating unit 9, the voxels can be removed by setting the voxel value of the voxels as 0. Also, when the image forming method referred to as the ray tracing or volume ray casting is used by the ultrasonic image generating unit 9, since the transparency for each voxel can be treated, the voxels can be removed by setting the transparency of the voxels.

The ultrasonic image generating unit 9 forms the image of the fetal surface 64 by 2-dimensionally projecting the volume data from which the voxels have been removed, and the display unit 10 displays the formed image of the fetal surface 64.

As described above, in accordance with the ultrasonic diagnostic apparatus in the present embodiment, the feature values which represent the feature of the voxels is calculated by generating an ultrasonic image from the determined voxels based on the gradient of the ultrasonic beam directions and the voxel values and characterizing the gradients of the voxel values by the ultrasonic beam directions, whereby making it possible to render an image of the fetal surface 64 with a small amount of calculation.

Also when a fetus grows in the uterus as the pregnancy progresses, the fetal surface 64 (border C) and the endometrial membrane (border B) starts coming into contract. Even in such a case, the fetal surface 64 can be distinguished by the ultrasonic diagnostic apparatus in the present embodiment. That is, the ultrasonic diagnostic apparatus in the present embodiment is capable of appropriately removing the region in which the fetal surface 64 (border C) and the endometrial membrane (border B) come into contact, whereby making it possible to render an image of the fetal surface 64.

In concrete terms, the ultrasonic reflected signals from the region in which the fetal surface 64 (border C) and endometrial membrane (border B) come into contract become very weak because no amniotic fluid is included therein, thus absolute value (vector length) |v| of the gradient which is calculated in the gradient calculating section 801 becomes small, and the region becomes included in distribution region F shown in FIG. 10(c). On the other hand, even when the fetal surface 64 and the endometrial membrane come into contact, since the ultrasonic reflected signals reflected in a fetal cranium which is equivalent to the fetal surface 64 are more intense than the ultrasonic reflected signals reflected in the surrounding tissue, the voxel values of the fetal cranium become larger values than the voxel values in the surrounding tissues, thus absolute value |v| of the gradient in the fetal cranium calculated in the gradient calculating section 801 become larger than that in the surrounding tissue. This vector length |v| of the fetal cranium is included in distribution region C shown in FIG. 10(c), the fetal cranium surface which is equivalent to the fetal surface 64 can be distinguished. Therefore, even when the fetal surface 64 (border C) and the endometrial membrane (border B) come in contact, the fetal surface 64 can be appropriately distinguished.

Also, by providing the operation unit 2 with devices such as a variable dial for respectively adjusting threshold values T1˜T3 and GUI, it is possible to adjust the accuracy in distinguishing the fetal surface 64.

### Second Embodiment

The ultrasonic diagnostic apparatus in Embodiment 2 related to the present invention will be described below referring to the attached diagrams. Unless specifically mentioned, other configuration is the same as that of the ultrasonic diagnostic apparatus in Embodiment 1.

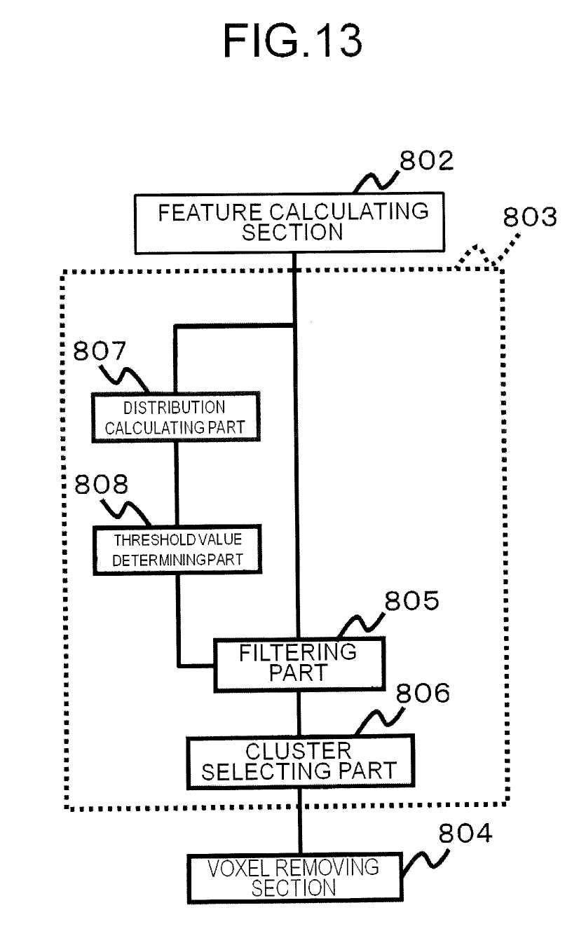

FIG. 13 shows the configuration of the object-voxel determining section 803 in the present embodiment.

The object-voxel determining section 803 comprises a distribution calculating part 807 and a threshold value determining part 808. The distribution calculating part 807 calculates the distribution of the vector lengths and vector directions of the gradient vectors in a feature space on the basis of the feature values calculated by the feature calculating section 802. In the present embodiment, the frequency distribution calculating part 807 calculates the frequency distribution categorized by vector length |v| of the gradient vectors and the frequency distribution categorized by the vector direction w·u. The threshold value determining part 808 determines threshold values T1 and T2 to be used in the filtering part 805 based on the distribution of the vector lengths and vector directions calculated by the distribution calculating part 807. The threshold value determining part 808 transmits the determined threshold values T1 and T2 to the filtering part 805.

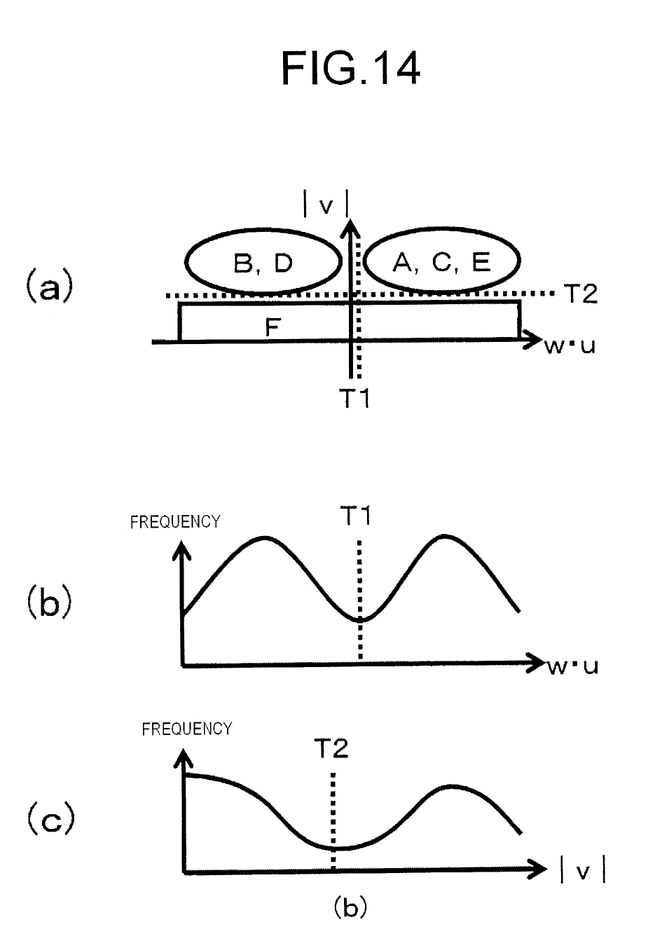

Next, the operation of the distribution calculating part 807 and the threshold value determining part 808 will be described referring to FIG. 14. FIG. 14(a) shows the distribution of vector lengths |v| and vector directions w·u in a feature space. FIG. 14(b) shows the frequency distribution of vector length |v| categorized by vector direction w·u. FIG. 14(c) shows the frequency distribution of vector direction w·u categorized by vector length |v|.

The distribution calculating part 807 calculates the distribution of vector lengths |v| and vector directions w·u in a feature space as shown in FIG. 14(a). Since the vector directions of the gradients in borders A, C and E shown in FIG. 9 are in the direction of ultrasonic beam b (depth direction) in FIG. 14(a), vector directions w·u in borders A, C and E are mainly distributed in the distribution regions having the value of 0 or above. Also, since borders B and D are in the direction opposite to ultrasonic beam b (depth direction), vector directions w·u of borders B and D are mainly distributed in the distribution regions having the value of 0 or below. Further, since vector lengths |v| of the gradient vectors in region F are short compared to those in borders A˜E and the variability of vector directions w·u is great, region F is distributed as shown in FIG. 14(a).

The threshold value determining part 808 determines threshold value T1 for distinguishing distribution regions A, C and E and distribution regions B and D, and determines threshold value T2 for distinguishing distribution regions A˜E and distribution region F as shown in FIG. 14(a). For example, the binarization process can be used as the method for determining threshold values T1 and T2. As shown in FIGS. 14(b) and (c), since the frequency distribution of vector length |v| and vector direction w·u in a feature space indicates the bimodal distribution having two peaks each, the value at which the ratio between the interclass variance and the intra-class variance reaches the maximum can be respectively determined as threshold values T1 and T2. Also by calculating the inclination of the curve in the frequency distribution as shown in FIGS. 14(b) and (c) by the first-order differential, the portions in which the inclination between the two peaks change from the negative to the positive may be determined as threshold values T1 and T2.

The determined threshold values T1 and T2 are transmitted to the filtering part 805, and the filtering part 805 selects the voxels that are in the distribution region in which vector direction w·u is larger than threshold value T1 and vector length |v| is larger than threshold value T2 based on the distribution in the feature space of vector length |v|, vector direction w·u and depth r, as shown in FIGS. 10(b) and (c).

In this manner, it is possible to determine threshold values T1 and T2 by providing the distribution calculating part 807 and the threshold value determining part 808.

### Embodiment 3

The ultrasonic diagnostic apparatus in Embodiment 3 related to the present invention will be described below referring to the diagrams. Unless specifically mentioned, other configuration is the same as that of the ultrasonic diagnostic apparatus in Embodiments 1 and 2. The ultrasonic diagnostic apparatus in the present embodiment comprises a device for setting the operand range of the gradient in three dimensions (operand range setting section), and the gradient calculating section 801 calculates the 3-dimensional gradient on the basis of the set operand range.

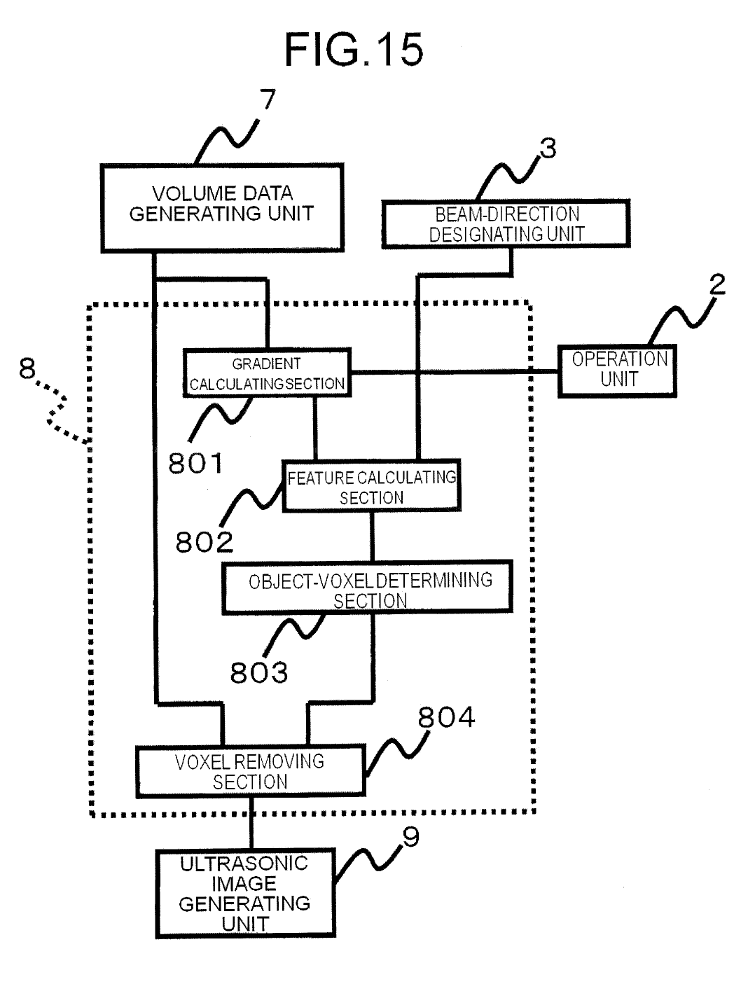

FIG. 15 shows the volume data processing unit 8 in the present embodiment. The gradient calculating section 801 in the volume data processing unit 8 is connected to the operation unit 2. The operation unit 2 changes the operand range of the operation to be used by the gradient calculating section 801 for calculating the gradient.

Next, the operation of the operation unit 2 for changing the operand range of an operation will be described. When an image of a fetal surface is generated from the volume data, a noise may appear in the vicinity of the fetal surface. Here, a noise is referred to structural objects which end up being displayed as a part of the fetal surface such as variegated acoustic interference referred to as an acoustic noise or a speckle, multiple echo and intra-amniotic fluid floatage. Since a noise appears near the fetal surface and has a strong ultrasonic reflected signal, the gradient in the portion at which the noise appears is mainly included in distribution region C of the feature space shown in FIG. 10(b) and (c). Also, the noise is localized in the region which is smaller than the fetal surface. By using the localization of the noise, the gradient calculating unit 801 calculates the gradient not to be included in distribution region C in the feature space shown in FIG. 10(b) and (c).

In order to calculate the gradient not to be included in distribution region C, the operand range of the operation is changed by the operation unit 2. In this manner, the gradient is calculated using the operation having the property that vector length |v| of the localized noise becomes small and vector length |v| in the vicinity of the fetal surface is unlikely to be small.

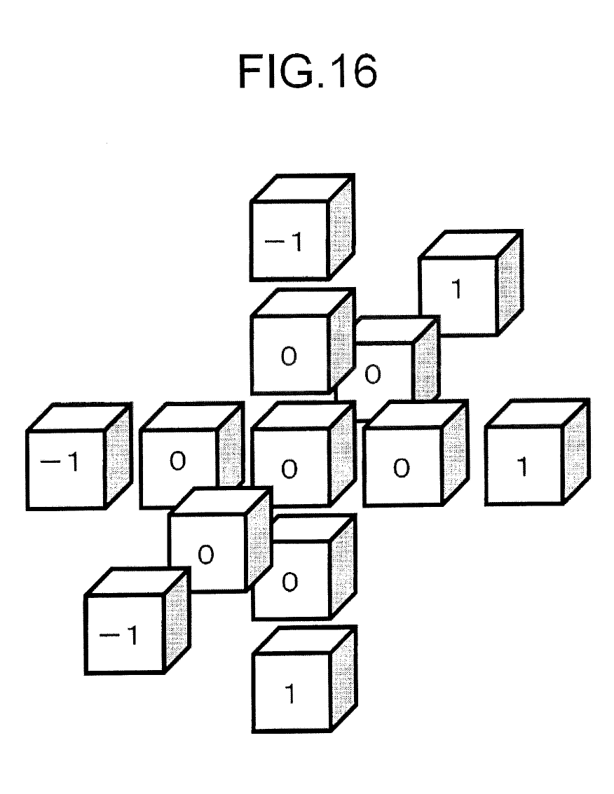

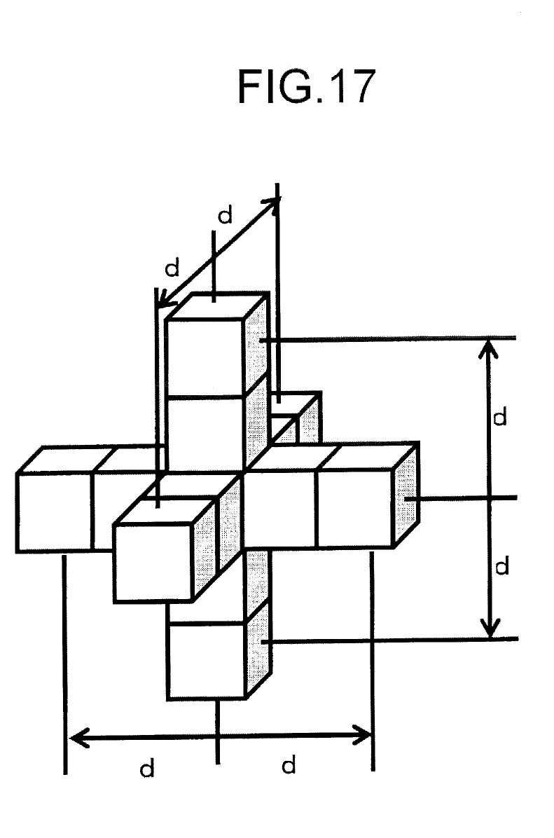

FIG. 16 shows the operand range of the operation which is adjusted by the operation unit 2. The operand range of the operation shown in FIG. 16 is wider than the operand range shown in FIG. 8(b). In other words, the operand range in each coordinate axis is widened by two voxels compared to that of FIG. 8(b). By calculating the gradient in target voxels using such an operation, vector length |v| of the gradient is reduced with respect to the localized noise, and the reduction rate of vector length |v| in the fetal surface can be made small compared to the reduction rate of vector length |v| in the noise, whereby making it possible to selectively identify a large structural object such as the fetal surface. In other words, while a noise is included in distribution region C in the feature space shown in FIG. 10(b) and (c) when the gradient of a noise is calculated by the operation shown in FIG. 8(b), the noise can be canceled by calculating it using the operation shown in FIG. 16 so that the noise is included in distribution region F in the feature space shown in FIG. 10(b) and (c) to be removed with distribution region F. FIG. 17 is a view showing that the operand range of an operation is variable. In the diagram, the operand range of an operation is indicated by d. An operand range d is transmitted from the operation unit 2 which is connected to the gradient calculating section 801. The operand range d is set as 1 in the operation shown in FIG. 8(b), and d is set as 2 in the operation shown in FIG. 16. By changing d to the value which is larger than 1, the operand range of the operation can be widened to the region which is respectively apart by d in the front and back, right and left, and top and bottom of the coordinate axis. In this manner, a large structural object such as a fetal surface can be selectively identified by changing d, which makes it possible to remove a structural object which is smaller than the fetal surface (a noise, etc.) and to remove a noise which interferes the generation of a smooth fetal surface image.

The preferable embodiments according to the present invention have been described above. However, the present invention is not limited to these embodiments, and various kinds of alterations or modifications can be made by persons skilled in the art within the scope of the technical idea disclosed in this application.

For example, while vector length |v| of the gradient, vector direction w·u of the gradient and voxel depth r are used as the feature values in the above-described embodiments, at least one of vector length |v|, vector direction w·u and voxel depth r may also be used as the feature values.

A case in which vector direction w·u of gradients and voxel depth r are set as the feature values will be described referring to FIG. 10. As shown in FIG. 10(b), when distribution regions A˜F are distributed in the feature space of vector direction w·u and voxel depth r, distribution regions A, C and E and a part of distribution region F are selected by the filtering part 805. In this case, threshold value T1 which is determined from the distribution of vector directions w·u in a feature space may also be used as shown in FIG. 14(b).

Then as shown in FIG. 11, the cluster selecting part 806 determines the cluster (distribution region C) as the voxels corresponding to the fetal surface 64 (border C) based on the frequency distribution of the distribution region categorized by voxel depth r (frequency distribution of vector directions w·u). In a case in which vector direction w·u and voxel depth r are set as the feature values, while a part of distribution region F is included in addition to distribution region C in the cluster selected by the cluster selecting part 806, the voxels corresponding to the fetal surface 64 (border C) can be determined based on the feature space of vector direction w·u and voxel depth r by adjusting operand range d of the operation for calculating the gradient and removing distribution region F. In this case, it is preferable to set operand range d as 2 or above.

A case in which vector length |v| of the gradient and voxel depth r are set as the feature values will be described referring to FIG. 10. As shown in FIG. 10(c), when distribution regions A˜F are distributed in the feature space of vector length |v| and voxel depth r, distribution regions A, B, C, D and E are selected by the filtering part 805. In this case, threshold value T2 which is determined from the distribution of vector length |v| in the feature space may also be used as shown in FIG. 14(c).

Then as shown in FIG. 11, the cluster selecting part 806 removes the clusters (distribution regions A and B) having the variation value which is smaller than threshold value 3 based on the frequency distribution of the distribution region categorized by voxel depth r (frequency distribution of vector length |v|), and selects the clusters (distribution regions C, D and E) having the variation value which is larger than threshold value T3. As described above, the vectors in border A and border B are in the direction which is approximately the same as the direction of ultrasonic beam b with comparatively small variability, thus the variation values in distribution regions A and B become comparatively small which is smaller than threshold value T3, and are removed by the cluster selecting part 806.

Then an image of the fetal surface 64 (border C) can be created, by rendering the very front surface in the line of sight from among distribution regions C, D and E that are selected by the cluster selecting part 806. For rendering the very front surface in the line of sight, a known rendering method such as volume ray casting or ray tracing can be applied.

A case in which vector direction w·u and vector length |v| of the gradient are set as the feature values will be described referring to FIG. 14. As shown in FIG. 14, threshold values T1 and T2 are determined, and the filtering part 805 selects distribution regions A, C and E on the basis of threshold values T1 and T2. Then a region of interest (ROI) is set in the region which is estimated as a fetus, and distribution region A which is comparatively shallow region is removed.

In this case, since distribution region B of border B which is in the vicinity of the fetal surface 64 (border C) is already removed, the ROI can be easily set in the region which is estimated as the fetus. By rendering the very front surface in the line of sight from among distribution regions C and E which remained after removal of distribution region A, an image of the fetal surface 64 (border C) can be created.

Also, by using the property such as the comparatively small variability of vector directions w·u in border A and border B and uniformity in vector lengths |v| in border A and border B, by using vector direction w·u and/or vector length |v| as the feature values, the fetal surface 64 (border C) can be depicted by rendering the very front surface in the line of sight from the remained distribution regions after appropriate removal of distribution regions A and B.

In this manner, at least one or two of vector length |v|, vector direction w·u and voxel depth r may also be used as the feature value.

Also, while the first-order differential or binarization process is used for distinguishing the frequency distribution of distribution regions in the above-described embodiments, other methods for distinguishing the frequency distribution of distribution regions may also be used such as using the portion having the minimum value between the peaks in the frequency distribution. Also, while the index of distribution is represented by the variance value in the above-described embodiments, other values such as standard deviation or average deviation may also be used.

Also, while the frequency distribution is used in the above-described embodiments, other methods for distinguishing the distribution of the feature values in a feature space may also be used.

### INDUSTRIAL APPLICABILITY

The ultrasonic diagnostic apparatus in the present invention is effective in rendering a surface image of an object with a small amount of calculation by generating an ultrasonic image from determined voxels based on the direction of the ultrasonic beam and gradients of the voxel values, calculating the feature values which represent the feature of the voxels by characterizing gradients of the voxel values by the direction of the ultrasonic beam and determining the voxels of the object based on the feature values in a feature space, in particular as the ultrasonic diagnostic apparatus, etc. for rendering an image of a fetal surface.

### DESCRIPTION OF REFERENCE NUMERALS

1 ultrasonic diagnostic apparatus
2 operation unit
3 beam direction instructing unit
4 transmitting/receiving unit
5 probe
7 volume data generating unit
8 volume data processing unit
9 ultrasonic image generating unit
10 display unit
801 gradient calculating section
802 feature calculating section
803 object-voxel determining section
804 voxel removing section
805 filtering part
806 cluster selecting part
807 distribution calculating part
808 threshold value determining part

1. An ultrasonic diagnostic apparatus comprising:
a volume data generating unit configured to generate volume data of an object to be examined by transmitting and receiving ultrasonic beams via a probe;
a volume data processing unit configured to generate an ultrasonic image of the object which is generated by the volume data generating unit; and
an ultrasonic image generating unit configured to generate the ultrasonic image corresponding to the object;
wherein the volume data processing unit is equipped with:
a gradient calculating section configured to calculate the gradient of the voxel values in the volume data;
a feature calculating section configured to calculate feature values of the voxel values on the basis of the gradient and the ultrasonic beam direction and calculate a feature space on the basis of the feature values;
an object-voxel determining section configured to determine the voxels corresponding to the object on the basis of the feature space; and
a voxel removing section configured to remove the voxels that are closer to the probe than the object.

2. The ultrasonic diagnostic apparatus according to claim 1, wherein the object-voxel determining section comprises a cluster selecting part configured to determine the voxels including the object based on the vector length and/or the vector direction of the gradients in the feature space.

3. The ultrasonic diagnostic apparatus according to claim 2, wherein the vector direction in the cluster selecting part is expressed by the inner product of the normalized vector of the ultrasonic beam and the normalized vector of gradients in the voxel values in the volume data.

4. The ultrasonic diagnostic apparatus according to claim 2, wherein:
the distribution in the cluster selecting part is the frequency distribution of the vector lengths or the vector directions categorized by the depth; and
the index of the distribution is represented by at least one of the variance value, standard deviation and average deviation on the basis of the frequency distribution.

5. The ultrasonic diagnostic apparatus according to claim 1, wherein the object-voxel determining section determines the voxels including the object by comparing a preset threshold value with the feature values.

6. The ultrasonic diagnostic apparatus according to claim 5, wherein the object-voxel determining section comprises:
a distribution calculating part configured to calculate the vector length and/or vector direction of the gradients in the feature space; and
a threshold value determining part configured to determine the threshold value on the basis of the distribution.

7. The ultrasonic diagnostic apparatus according to claim 1, wherein the feature calculating section calculates a feature space in which at least one of the vector length and vector direction of gradients in the volume data voxel values and the depth of the voxels is set as the feature value.

8. The ultrasonic diagnostic apparatus according to claim 1, wherein the voxel removing section sets the voxel value of the voxels that are positioned on the probe side as a predetermined value.

9. The ultrasonic diagnostic apparatus according to claim 1, wherein the voxel removing section sets the transparency of the voxels that are positioned on the probe side.

10. The ultrasonic diagnostic apparatus according to claim 1, wherein the gradient calculating section calculates the gradients in three dimensions on the basis of an operation, and the operand range of the operation is variable.

11. The ultrasonic diagnostic apparatus according to claim 1, comprising a device for setting the operand range of the gradients in three dimensions, wherein the gradient calculating section calculates the gradients in three dimensions on the basis of the set operand range.

12. An ultrasonic image rendering method for generating an ultrasonic image of an object to be examined from the volume data acquired by an ultrasonic diagnostic apparatus provided with a probe, including:
calculating gradients of voxel values in the volume data;
calculating feature values of voxels based on the vector directions of the gradients and the gradients of the voxel values, and calculating a feature space on the basis of the feature values;
determining the voxels corresponding to the object on the basis of the feature space;
removing the voxels that are closer to the probe than the object; and
generating an ultrasonic image corresponding to the object from the volume data from which the voxels that are positioned on the probe side have been removed.

13. The ultrasonic image rendering method according to claim 12, wherein the determination of the voxels comprises selecting of a cluster which determines the voxels including the object on the basis of the distribution of the vector lengths and/or the vector directions of the gradients in the feature space.

14. The ultrasonic image rendering method according to claim 12, wherein the determination of the voxels determines the voxels including the object by comparing a preset threshold value and the feature values.

15. The ultrasonic image rendering method according to claim 12, wherein the calculation of the feature space calculates a feature space in which at least one of the vector length and vector direction of gradients in the volume data voxel values and the depth of the voxels is set as the feature values.
